(19) United States
(12) Patent Application Publication
(10) Pub. No.: US 2012/0149101 A1
(43) Pub. Date: Jun. 14, 2012

(54) METHODS AND KITS FOR REGULATING INTRACELLULAR TRAFFICKING OF A TARGET PROTEIN

(75) Inventor: Franck Perez, Paris (FR)

(73) Assignee: INSTITUT CURIE, Paris 5 (FR)

(21) Appl. No.: 13/377,278

(22) PCT Filed: Jun. 11, 2010
(86) PCT No.: PCT/EP10/58229

§ 371 Date: Dec. 9, 2011

(30) Foreign Application Priority Data
Jun. 11, 2009 (EP) 09305534.1

(51) Int. Cl. C12N 15/63; C12N 1/19; C12N 5/10

(52) U.S. Cl. 435/348; 435/325; 435/254.2; 435/320.1

(57) ABSTRACT

A method and kits for regulating the intracellular trafficking of a target protein. In a retained state, the target protein is retained in a first compartment by an interaction with a hook protein. In a released state, the interaction is disrupted and the target protein traffics to a target compartment.

### FIELD OF THE INVENTION

The invention relates to a method and to kits for regulating the intracellular trafficking of a target protein.

### BACKGROUND OF THE INVENTION

The Golgi complex plays a central role in eukaryotic cell homeostasis. It processes and sorts proteins and lipids synthesized in the endoplasmic reticulum (ER) and serves as a central platform connecting the anterograde and retrograde trafficking pathways. These activities are coupled to unique ultrastructural characteristics. The Golgi apparatus is composed of stacks of flattened, adherent cisternae (Rambourg and Clermont, 1997; Ladinsky et al., 1999) that display a cis to trans polarity. In certain eukaryotes, and in particular in humans, hundreds of stacks are laterally connected to form an extended ribbon-like structure next to the microtubule organizing centers.

Despite the large and continuous flow of membranes and proteins occurring at steady state, the overall organization, ultrastructural shape and polarity of the Golgi apparatus is remarkably stable. Each Golgi cisternae contains a particular set of “resident” proteins, such as glycosylation enzymes, but how this is maintained has been debated (Martinez-Menarguez et al., 2001; Cosson et al., 2002; Altan-Bonnet et al., 2004; Puthenveedu and Linstedt, 2005; Storrie, 2005). Two extreme models have been proposed to explain how such a structure is dynamically maintained. According to the “static cisternae” model, cargo advances, packed in vesicles or using extended tubules, through a stable stacked structure (Pelham, 2001). Resident proteins are stably localized to particular cisternae using specific signals, through interactions with the membranes or with the matrix. The Golgi matrix has been proposed to be stable and inheritable exoskeleton that may serve as a template for the maintenance of the Golgi complex. This matrix is particularly important upon mitosis exit (Shorter and Warren, 2002) but is also proposed to play a role during interphase to maintain Golgi structure (reviewed in Glick, 2002). According to the “cisternal maturation” model, the Golgi apparatus in endowed with auto-organization abilities and does not depend on an external matrix to build and maintain its structure. Cargoes are transported inside maturating cisternae and resident proteins achieve their steady-state localization through retrograde transportation. (reviewed in Glick, 2002; Shorter and Warren, 2002; Barr, 2004). Independently of the model, inter-cisternae transport (respectively of cargo or of resident proteins) may occur via vesicular or tubular connections (for reviews see Mironov et al., 2005; Rabouille and Klumperman, 2005). Recent models even suggest that intra-Golgi transport is done through very fast tubule-based diffusion (Patterson et al., 2008)

It is now clear that multiple pathways cross the Golgi apparatus and this has both fundamental and applied consequences. Molecular studies of various pathologies, and above all cancer, have identified receptors and hormones as key regulators of disease development. These proteins may follow particular secretory routes. In addition, the large scale genomic projects have identified many proteins potentially involved in the regulation of the secretory pathway, proteins that have yet to be functionally characterized. Annotating functionally these large families of proteins may thus on the one hand help to identify new therapeutic entries and on the other hand help us to draw a general map of cellular secretory pathways.

To get a comprehensive view of the multiple secretory pathways, it is thus essential to widen our collection of trafficking and secretory assays. Only few methods exist in the art that can bring quantitative data and that can be adapted to large scale projects. However, they suffer from various drawbacks.

The best methods used to quantify the trafficking of a target protein all rely on the synchronization of the secretion of all the molecules of said target protein in a cell, in order to have an observable read-out at the population level.

During the last two decades, many studies have extended significantly our understanding of membrane protein sorting in the secretory pathway using as a model a temperature-sensitive variant of the transmembrane glycoprotein of the stomatitis virus (VSVG-ts045). This mutant is blocked in the ER at high temperature (39.5° C.) and transported to the plasma membrane at the permissive temperature (32° C.) (Lafay, 1974; Kreis, 1986). This model is powerful because it allows the synchronous transport and processing of VSVG through the secretory pathway by simple temperature shift. This synchronized trafficking can also be studied in living cells (Arnheiter et al., 1984; Presley et al., 1997; Scales et al., 1997).

However, this powerful system is hindered by several limitations. The high temperature of 39.5° C. is not physiological (nor is the lower 32° C. permissive temperature). It can induce irreparable damages and it not usable in other multi-cellular organisms such as D. melanogaster and C. elegans for example. Secondly, using the VSVG-ts045 only the trafficking of one category of protein, bearing a single transmembrane domain, can be analyzed. Some results however suggest that some categories of proteins, such as soluble proteins and GPI-anchored proteins, destined to the cell surface, are segregated from the VSVG-ts045 in the Golgi apparatus (Keller et al., 2001). Thirdly, the VSVG-ts045 allows only the study of the secretory pathway from the ER to plasma membrane. It does not allow the study of the transport to the endosomes and lysosomes for example, or to certain plasma membrane domains like the apical membrane or the axon. Moreover, the studies of intermediate steps of the secretory pathway (like the intermediate compartment to Golgi or trans-Golgi Network to plasma membrane) can only be performed using mysterious temperature blocks.

Other studies have used an inducible promoter in order to synchronize the production and secretion of a target protein. Indeed, Bard et al. (2006) studied the secretion of horseradish peroxidase fused to a signal sequence under the control of a Cu²⁺ inducible promoter. The induction using this system is rather slow and depends on a set of pathways (transcription, translation, translocation in the ER) that complicates analysis. This also only allows studying the trafficking from the ER.

Yet other studies rely on the use of photoconversion and/or photobleaching of a population of molecules of the target protein. However, the major drawback of this method is that it requires a cell-by-cell analysis, which is not suitable for screening purposes. Also, adverse effects that perturb the cellular physiology can be provoked by the photoconversion or photobleaching treatments.

Another method for regulating the trafficking of a given target protein has been proposed in patent application WO00/23602 to Ariad Gene Therapeutics. This document discloses a method for genetically engineering cells to be capable of regulated secretion of a target protein comprising introducing into a cell a recombinant nucleic acid encoding a fusion protein comprising at least one conditional retention domain and at least one additional domain that is heterologous thereto. Said a conditional retention domain is typically a conditional aggregation domain (CAD), i.e. a protein that aggregates in a small molecule reversible manner. This technology was used for example for the regulation of the secretion of target proteins such as insulin and growth hormone (Rivera et al. 2000). In this study; the authors created a fusion protein that includes a CAD (a domain that interacts with itself in the absence of a ligand and is thus retained in the ER in the absence of ligand) and the target protein.

This technique allows the controlled secretion of secreted proteins in vivo by addition of a small molecule. However, since it relies on a reversible aggregation, it only allows the study of trafficking from the ER as a donor compartment. It cannot be used for studying trafficking from other intracellular compartments, in particular for studying retrograde trafficking. In addition, this aggregation system may induce the unfolded protein response pathway which would influence cell physiology.

Thus, there is still an unanswered need in the art for a versatile method, both amenable to high-throughout and to living cells analysis, for studying the trafficking of a target protein in a host cell by allowing the fast and synchronous release of said target protein.

### SUMMARY OF THE INVENTION

Thus, the inventors have set up a new system to study the secretory pathway of proteins. They have called it RUSH (Retention Using Selective Hooks) as it is based on the selective retention and release of cargo. The principle of the method is rather generic. It provides a target protein in two states: “retained”, i.e. blocked in the donor compartment by a specific interaction with a resident protein, the hook, and “released” from the interaction, free to traffic toward its target compartment. To control these two states, the specific interaction between the target protein and the hook is mediated by a reversible interaction between two interaction domains. In one embodiment, the interaction only occurs in the presence of a given ligand (“molecule-dependant” set-up, “MD”). In another embodiment, the interaction occurs by default and can be disrupted by a given ligand (“interaction-by-default” setup, “ID”).

The removal or addition of the ligand acts like a switch to allow the synchronous release of the target protein from the donor compartment.

The invention relates to a method for regulating the intracellular trafficking of a target protein Y in a host cell comprising:

a) providing a host cell;
b) providing a first expression vector comprising a nucleotide sequence encoding a first fusion protein of formula A-X, wherein A is an interaction domain and X is a domain capable of retaining the first fusion protein of formula A-X in a given intracellular compartment;
c) providing a second expression vector comprising a nucleotide sequence encoding a second fusion protein of formula B-Y, wherein B is an interaction domain and Y is the target protein;

wherein A and B are capable of a conditional interaction according to the presence or absence of a ligand L.

The invention also relates to a kit for regulating the intracellular trafficking of a target protein Y in a host cell comprising:
a first expression vector comprising a nucleotide sequence encoding a first fusion protein of formula A-X wherein A is an interaction domain and X is a domain capable of retaining the first fusion protein of formula A-X in a given compartment and
a second expression vector comprising a nucleotide sequence encoding a second fusion protein of formula B-Y, wherein B is an interaction domain and Y is the target protein;
   wherein A and B are capable of a conditional interaction according to the presence or absence of a ligand L.

The invention also relates to the use of:
a first expression vector comprising a nucleotide sequence encoding a first fusion protein of formula A-X, wherein A is an interaction domain and X is a domain capable of retaining the first fusion protein of formula A-X in a given intracellular compartment;
and a second expression vector comprising a nucleotide sequence encoding a second fusion protein of formula B-Y, wherein B is an interaction domain and Y is the target protein;
   wherein A and B are capable of a conditional interaction according to the presence or absence of a ligand L;
   for selectively retaining and releasing a target protein Y from a donor compartment.

In a preferred embodiment, the synchronous release of the target protein from the donor compartment is controlled by addition of a ligand L, which disrupts the interaction between A and B.

### DETAILED DESCRIPTION OF THE INVENTION

In one aspect, the invention relates to a method for regulating the intracellular trafficking of a target protein Y in a host cell comprising:

a) providing a host cell;
b) providing a first expression vector comprising a nucleotide sequence encoding a first fusion protein of formula A-X, wherein A is an interaction domain and X is a domain capable of retaining the first fusion protein of formula A-X in a given intracellular compartment;
c) providing a second expression vector comprising a nucleotide sequence encoding a second fusion protein of formula B-Y, wherein B is an interaction domain and Y is the target protein;
wherein A and B are capable of a conditional interaction according to the presence or absence of a ligand L.

As used herein, the expression “regulating the intracellular trafficking of a target protein Y in a host cell” refers to the fact of controlling the intracellular localization of the target protein according to the presence or absence of a ligand, L. In a retained state, the target protein Y is retained in a given intracellular compartment. Upon addition or removal of the ligand, the target protein Y is released. The release of said target protein Y is fast and synchronous for all the molecules of the target protein Y.

Therefore, the invention relates to a method for regulating the intracellular trafficking of a target protein Y in a host cell by allowing the synchronous release of said target protein Y, said method comprising:

a) providing a host cell;
b) providing a first expression vector comprising a nucleotide sequence encoding a first fusion protein of formula A-X, wherein A is an interaction domain and X is a domain capable of retaining the first fusion protein of formula A-X in a given intracellular compartment;
c) providing a second expression vector comprising a nucleotide sequence encoding a second fusion protein of formula B-Y, wherein B is an interaction domain and Y is the target protein;
wherein A and B are capable of a conditional interaction according to the presence or absence of a ligand L.

The invention also relates to a method for regulating the intracellular trafficking of a target protein Y in a host cell comprising:

a) providing a host cell;
b) providing a first expression vector comprising a nucleotide sequence encoding a first fusion protein of formula A-X, wherein A is an interaction domain and X is a domain capable of retaining the first fusion protein of formula A-X in a given intracellular compartment;
c) providing a second expression vector comprising a nucleotide sequence encoding a second fusion protein of formula B-Y, wherein B is an interaction domain and Y is the target protein;
wherein A and B are capable of a conditional interaction according to the presence or absence of a ligand L;
d) releasing the target protein Y by removal or addition of said ligand L, respectively;
e) optionally, analyzing the intracellular trafficking of the target protein Y at different time points after step d).

The method of the invention has many advantages: 1) it avoids the use of temperature blocks; 2) it allows to study a large set of trafficking steps; 3) it is applicable to kinetic and quantitative studies; 4) it allows to study the secretory pathway of a variety of reporter molecules and to understand the mechanisms and signals implicated in their delivery to their final destination; 5) this system is amenable to High Throughput screening. This opens the possibility of screening large siRNA libraries. This is particularly important in this post-genome era where a lot of potential regulator of intracellular trafficking have been identified but need to be annotated (like the Golgi matrix proteins). Importantly, chemical libraries can also be screened using this assay to find specific inhibitors or enhancers of specialized pathways and in particular pathways transporting molecules involved in human diseases like cancer (e.g. EGFR, HER2, VEGF) or virus infection (e.g. HIV).

In a preferred embodiment, the synchronous release of the target protein from the donor compartment is controlled by addition of a ligand L, which disrupts the interaction between A and B.

The invention therefore relates to a method for regulating the intracellular trafficking of a target protein Y in a host cell comprising:

a) providing a host cell;
b) providing a first expression vector comprising a nucleotide sequence encoding a first fusion protein of formula A-X, wherein A is an interaction domain and X is a domain capable of retaining the first fusion protein of formula A-X in a given intracellular compartment;
c) providing a second expression vector comprising a nucleotide sequence encoding a second fusion protein of formula B-Y, wherein B is an interaction domain and Y is the target protein;
wherein A and B are capable of a conditional interaction in the absence of a ligand L;
d) releasing the target protein Y by addition of said ligand L.

In one aspect, the invention also relates to the use of:
a first expression vector comprising a nucleotide sequence encoding a first fusion protein of formula A-X, wherein A is an interaction domain and X is a domain capable of retaining the first fusion protein of formula A-X in a given intracellular compartment;
and a second expression vector comprising a nucleotide sequence encoding a second fusion protein of formula B-Y, wherein B is an interaction domain and Y is the target protein;
   wherein A and B are capable of a conditional interaction according to the presence or absence of a ligand L;
   for selectively retaining and releasing a target protein Y from a donor compartment.

Also described herein are kits suitable for carrying out the method of the invention.

The invention therefore also relates to a kit for regulating the intracellular trafficking of a target protein Y in a host cell comprising:
a first expression vector comprising a nucleotide sequence encoding a first fusion protein of formula A-X wherein A is an interaction domain and X is a domain capable of retaining the first fusion protein of formula A-X in a given compartment and
a second expression vector comprising a nucleotide sequence encoding a second fusion protein of formula B-Y, wherein B is an interaction domain and Y is the target protein;
   wherein A and B are capable of a conditional interaction according to the presence or absence of a ligand L.

As indicated above, the expression “regulating the intracellular trafficking of a target protein Y in a host cell refers to the fact of allowing the synchronous release of said target protein from a given intracellular compartment.

In one embodiment, the kit further comprises an explanation leaflet which explains that the components of the kits are useful for allowing the synchronous release of a target protein Y from a donor compartment and for the subsequent analysis of the intracellular trafficking of said target protein Y.

Different techniques, known to the skilled person in the art, can be used (such as, but not limited to fluorescence microscopy, electron microscopy and biochemical analysis after cell fractionation).

In one embodiment, the kit further comprises a host cell capable of being transfected with said first and second expression vectors.

In one embodiment, the kit further comprises a transfection reagent.

Said transfection reagent can be selected from the many available transfection reagents in the art.

Suitable transfection reagents can be for example Lipofectamin 2000 (Invitrogen), Fugene 6 (Roche) or a simple Calcium Phosphate homemade solution.

In one embodiment, the kit further comprises a ligand L.

In one embodiment, the first expression vector comprises a nucleotide sequence encoding A and a multiple cloning site enabling the in-frame insertion of a nucleotide sequence encoding X in order to encode the first fusion protein A-X.

Advantageously, said kit allows for an exhaustive study of the trafficking of a given target protein Y from a variety of donor compartments, by varying the retention domain X.

In one embodiment, the second expression vector comprising a nucleotide sequence encoding B and a multiple cloning site for the in-frame insertion of Y in order to encode the second fusion protein of formula B-Y.

Advantageously, said kit allows for the study of the trafficking of a number of different target proteins Y from a given donor compartment, by varying the target protein Y, and using a given retention domain X.

In one embodiment, the kit comprises:
a first expression vector comprising a nucleotide sequence encoding A and a multiple cloning site enabling the in-frame insertion of a nucleotide sequence encoding X in order to encode the first fusion protein A-X; and
a second expression vector comprising a nucleotide sequence encoding B and a multiple cloning site for the in-frame insertion of Y in order to encode the second fusion protein of formula B-Y.

The expression “in-frame insertion” as used herein refers to the insertion, into a first nucleotide sequence encoding a first protein, of a second nucleotide sequence encoding a second protein in such a manner that the expression of the resulting nucleotide sequence results in the expression of a fusion between the first and second proteins. It falls within the ability of the person skilled in the art, starting from a given nucleotide sequence containing a multiple cloning site, to select the appropriate restriction enzymes and sequence to be inserted into said multiple cloning site.

Throughout this application, it is understood that, when referring to a fusion protein A-X, said fusion protein comprises the amino acid sequences of A and the amino acid sequence of X, in any given order. For example, X can be fused downstream of A, at its C-terminus, or A can be downstream of X. The fusion protein A-X can also comprise other amino acids than those defined by A and X. Said amino acids can be linker sequences, located between and A and X, and/or header sequences (at the N-terminus of both A and X) and/or tail sequences (at the C-terminus of both A and X). Similarly, the expression “fusion protein B-Y” covers any protein comprising the sequences of B and Y, whatever the configuration of said sequences.

As used herein, the terms “expression vector” refer to a nucleic acid molecule capable of directing the expression of a given nucleic acid sequence which is operatively linked to an expression control sequence or promoter. In particular, an expression vector according to the invention is a vector which enables the expression of a given nucleic acid sequence into the protein encoded by said nucleic acid sequence in a eukaryotic host cell. The promoter of said expression vector is typically a eukaryotic promoter.

The expression vector(s) of the present invention can be a plasmid or a viral vector. A plasmid is a circular double-stranded DNA loop that is capable of autonomous replication. A viral vector is a nucleic acid molecule which comprises viral sequences which can be packaged into viral particles. A variety of viral vectors are known in the art and may be adapted to the practice of this invention, including e.g., adenovirus, AAV, retrovirus, hybrid adeno-AAV, lentivirus and others. By carrying out routine experimentation, the skilled person in the art can chose from the variety of available vectors, those which are suitable for carrying out the method of the invention.

In a preferred embodiment, the first and second expression vector can be a single expression vector, said single vector comprising a bicistronic expression cassette. Vectors containing biscitronic expression cassette are well known in the art. Advantageously, bicistronic expression cassettes contain an Internal Ribosome Entry Site (IRES) that enables the expression of both fusion proteins from a single promoter. Thus, in this embodiment, the first fusion protein A-X and the second fusion protein B-Y are expressed at the same level in the host cell.

Suitable commercially available bicistronic vectors can include, but are not limited to plasmids of the pIRES (Clontech), pBud (Invitrogen) and Vitality (Stratagene) series.

In a preferred embodiment, the interaction domains A and B are distinct protein domains. In other words, the interaction between A and B, and therefore between A-X and B-Y, is a hetero-complex, rather than a homocomplex or auto-aggregate.

In one embodiment, the interaction between A-X and B-Y occurs at the luminal/exoplasmic face of the compartments (“luminal RUSH”, or RUSH^{L}). In another embodiment, the interaction between A-X and B-Y occurs at the cytoplasmic face (“cytoplasmic RUSH”, or RUSH^{C}).

In one embodiment, the interaction between A-X and B-Y occurs in a molecule-dependent way in the presence of a ligand L (“molecule-dependent” or “MD” set-up), and can be reversed by wash-out of the ligand L.

According to this embodiment, the interaction between A and B, and therefore between A-X and B-Y occurs only in the presence of a given ligand. This embodiment is called the “MD” mode.

Regulation of the interaction, which results in the release of the second fusion protein B-Y (comprising the target protein Y) from the second fusion protein A-X (comprising the Hook A), can be carried out by wash-out of the ligand L, with or without competition by competitor C, which competes with L for binding to either A or B, without inducing the interaction between A and B.

In a preferred embodiment, the MD interaction couple (A/B, or B/A) is FKBP-FK506 binding domain 12/FKBP-rapamycin associated protein (FKBP12/FRAP). FKBP12 (also known as FKBP1A) is a FK506 and rapamycin-binding protein of 12 kD (Standaert et al., 1990; Maki et al., 1990). FRAP is a 245 kD which binds to the FKBP12-rapamycin associated protein (Brown et al., 1994). In a preferred embodiment of the RUSH system, only the rapamycin-binding domains are used.

In this embodiment, the interaction occurs only in the presence of rapamycin or analogues thereof as a ligand L.

The ligand L can be any ligand able to mediate the interaction between FKBP12 and FRAP and can be, in particular, selected from the group consisting of FK1012, FK-CsA and rapamycin. Analogs of Rapamycin (Rapalog) may also be used in conjunction with mutants of FKBP12 and FRAP domains (like AP21967, ARIAD Pharmaceutical Inc.)

These ligands have been extensively used in systems for controlling gene expression at the transcriptional level (see Clackson 1997 for review).

Rapamycin (commercially available from Sigma-Aldrich for example) can be used at concentrations ranging from 1.5 nM to 200 nM, preferably from 1.52 nM to 12.2 nM, even more preferably at about 3.1 nM.

FK506 can be used as a competitor C and can therefore be added when rapamycin is removed, in order to disrupt the interaction between FKPB12 and FRAP. FK506 (commercially available from Cayman for example) can be used at concentrations ranging from 390 μM to 1.25 μM, preferably at about 3.3 μM. Other competitors can be used, such as Ascomycin (Sigma-Aldrich) at concentrations ranging from 12.5 μM to 1.6 μM, preferably at about 3.3 μM or SLF (Cayman) at concentrations ranging from 28.6 μM to 3.6 μM and preferably at about 5 μM.

Alternatively, the MD interaction couple (A/B, or B/A) is FKBP-rapamycin binding domain 12/a protein that binds to FKBP12 in a rapamycin-dependent manner. In this embodiment, the interaction occurs only in the presence of rapamycin or analogues thereof as a ligand L.

Document U.S. Pat. No. 6,492,106 discloses methods for identifying such proteins that bind to FKBP12 in a rapamycin-dependant manner.

In another embodiment, the interaction between A-X and B-Y occurs by default in the absence of ligand L (“interaction by default” or “ID” set-up) and is inhibited in the presence of a ligand L.

In this embodiment, the interaction between A and B, and therefore between A-X and B-Y, occurs by default, in the absence of any ligand. The interaction is disrupted by the presence of a ligand L.

Suitable ID interaction domain couples (A/B or B/A) can be selected for example from the group consisting of Streptavidin/SBP tag, Ftsz/ZipA, HPV E1/E2, recombinant antibody/epitope, recombinant epitope/hapten, proteinA/IgG domain, Fos/Jun. Interaction domain couples for which a molecule (ligand L) inhibiting the interaction is already known are preferred.

In one embodiment, the ID interaction domain couple (A/B or B/A) is FtsZ/ZipA. FtsZ and ZipA are bacterial proteins which form part of the septal ring which forms during the replication of certain Gram-negative bacteria. Their interaction can be disrupted by addition of a small molecule named “compound I” as a ligand L (see Wells et al. 2007 for review.).

Compound 1 (Wyeth Research (NY, USA)) can be used at concentrations ranging between 10 and 100 μM.

In another embodiment, the ID interaction domain couple (A/B or B/A) is streptavidin/SBP and free biotin is used as a ligand L. Streptavidin is a bacterial protein that binds with very high affinity to vitamin D-biotin. In vitro selection approaches have led to the discovery of synthetic peptides that bind to Streptavidin and that can be competed out by biotin.

A high affinity binder to Streptavidin, the SBP tag (as set forth in SEQ ID NO:1), has been identified by Wilson, Keefe and Szostak (2001) (see patent US 2002/0155578 A1):

SEQ ID NO: 1:
MDEKTTGWRGGHVVEGLAGELEQLRARLEHHPQGQREP.

A smaller version of Steptavidin, core Streptavidin have been defined in U.S. Pat. No. 5,672,691 (SEQ ID NO:2).

SEQ ID NO: 2:
MDPSKDSKAQVSAAEAGITGTWYNQLGSTFIVTAGADGALTGTYESAV
GNAESRYVLTGRYDSAPATDGSGTALGWTVAWKNNYRNAHSATTWSGQ
YVGGAEARINTQWLLTSGTTEANAWKSTLVGHDTFTKVKPSAASIDAA
KKAGVNNGNPLDAVQQ.

A monomeric core Streptavidin has also been constructed by Wu and Wong (2005) (see U.S. Pat. No. 7,265,205 B2 and SEQ ID NO:3).

SEQ ID NO: 3:
MDPSKDSKAQVSAAEAGITGTWYNQLGSTFIVTAGADGALTGTYESAV
GNAESRYTLTGRYDSAPATDGSGTALGWRVAWKNNYRNAHSATTWSGQ
YVGGAEARINTQWTLTSGTTEANAWKSTLRGHDTFTKVKPSAASIDAA
KKAGVNNGNPLDAVQQ.

As used herein, “Streptavidin” can refer to all forms of streptavidin (tetramer, core or monomer). In a preferred embodiment, streptavidin comprises the amino acid sequence as set forth in SEQ ID NO:2 or SEQ ID NO:3, or a variant thereof having at least 80% identity with SEQ ID NO:2 or SEQ ID NO:3, preferably 85%, 90, 95, 96, 97, 98, 99, 99.5% identity with SEQ ID NO:2 or SEQ ID NO:3.

“Streptavidin” can also encompass Streptavidin homologs from other species, such as avidin or rhizavidin. Mutant of these natural biotin-binding proteins may also be used.

Biotin can be used as a ligand L at concentrations ranging from 100 nM to 100 μM, preferably about 1 to 10 μM).

The retention domain X (or “Hook”) can be any protein or protein domain which is resident of a given intracellular compartment.

The term “resident”, when used herein applied to a given protein or domain and to a given compartment, is intended to mean that said protein or domain is in majority located in a given compartment. Typically, at least 70%, preferably at least 75%, 80%, 85%, 90%, 95%, 96%, 97%, 98%, 99% of said protein or domain is located in said compartment at steady-state in a host cell.

As used herein, the term “compartment” or “subcellular compartment” has its general meaning in the art of cell biology. It refers to a given subdomain of a eukaryotic cell. Typically, a compartment can be an organelle (endoplasmic reticulum, Golgi apparatus, endosome, lysosome, etc.), or an element of an organelle (multi-vesicular bodies of endosomes; cis-, medial- or trans-cisternae of the Golgi apparatus, etc.) or the plasma membrane or sub-domains of the plasma membrane (apical, basolateral, axonal, dendritic, etc.) or even microdomains (triton insoluble domains, focal adhesion, tight junctions, etc.).

As used herein, the expressions “donor compartment” and “acceptor compartment” have their general meaning in the art and relate to the compartment from which a given target protein originates and the compartment to which it is targeted, respectively.

According to the donor compartment of interest, different proteins or domains can be used as retention domains.

Suitable retention domains X in the ER are, but are not limited to, an isoform of the invariant chain which resides in the ER (Ii33), Ribophorin I or II (Strubin et al., 1986; Strubin et al., 1984; Schutze et al., 1994; Fu et al. 2000), SEC61, cytochrome b5 (Bulbarelli et al., 2002) or fragments thereof comprising the localization domains. An example of ER localization domain is the ER localization of Ribophorin II, available under Genbank accession number BC060556.1.

Suitable retention domains X in the Golgi apparatus are, but are not limited to, Giantin (GolgB1, GenBank Accession number NM_{—}004487.3), TGN38/46, Menkes receptor, and Golgi enzymes such as ManII (α-1,3-1,6 mannosidase, available under Genbank accession number NM_{—}008549), Sialyl Transferase (β-galactosamide α-2,6-sialyltranferase 1, NM_{—}003032), GalT (β-1,4-galactosyltransferase 1, NM_{—}001497) or fragments thereof comprising the localization domains.

Examples of plasma membrane retention domains X are, but are not limited to, GPI-anchored proteins such as Thy-1 and PRNP (Tanya et al., 2006; Schuck and Simons, 2006; Harris, 2003; Bard, et al., 2006; Hennecke and Cosson, 1993; Achour L, et al., 2009. Rayner and Pelham, 1997; Amaral, 2005).

In a preferred embodiment, the retention domain X is Ii33, an isoform of the invariant chain which resides in the ER.

The target protein Y according to the invention can be any protein for which is desirable to study the intracellular trafficking from a given donor compartment to a final target compartment.

Examples of target proteins Y can be, but are not limited to
growth factors such as Epidermal Growth Factor (EGF), Fibroblast Growth Factor (FGF), vascular endothelial growth factor (VEGF), all of which can be normal or mutated;
receptors, such as G-protein-coupled receptors (GPCRs) such as CCR5 and CFTR;
plasma membrane markers and Major HistoCompatibility (MHC) molecules such as CD4, CD8 and the model transmembrane protein TM21;
adhesion molecules such as E-cadherin;
lysosomal enzymes;
Golgi enzymes such as ManII (α-1,3-1,6 mannosidase), Sialyl Transferase (β-galactosamide α-2,6-sialyltranferase 1), GalT (β-1,4-galactosyltransferase 1);
viral glycoproteins such as VSVG and HA;
tetraspanning proteins such as CD9;
signal transduction proteins;
Transporter proteins like the multidrug resistance protein ABCB1;
Synthetic transmembrane domain (e.g. TMD22);
GPI-anchored proteins such as Thy-1 and Prp;
hormones (Insulin, Prolactin) or hormone receptors;
pathological molecules (amyloid peptide).

The target protein Y can be any molecule of therapeutic interest, for which it is desirable to tightly regulate the intracellular trafficking in order to obtain a therapeutic effect. Conversely, the target protein Y can be a pathological molecule, whose pathological effect is linked to its intracellular trafficking.

In a preferred embodiment, the target protein Y is selected from the group consisting of Sialyl Transferase, E-Cadherin and TMD22.

Sialyl Transferase and E-Cadherin are preferred target proteins for the RUSH^{L} set-up, whilst TMD22 is a preferred target protein for the RUSH^{C} set-up.

A given protein can in some embodiments be a retention domain X or a target protein Y, depending on the relative to the strength of retention. A given protein P1 may be more stably retained at its proper location that protein P2 and will this be considered as a retention domain or Hook (X). The same protein P1 may be less strongly retained that protein P3. Protein P3 will bring protein P1 to the final compartment of protein P3. P3 will be the retention domain or Hook (X) in this case.

Accordingly, the method of the invention can also be used to “rank” the strength of different localization domains.

Detection of the target protein Y can be carried out by any means known to the person skilled in the art.

In one embodiment, the target protein Y comprises a detectable moiety Z.

In another embodiment, the second fusion protein comprises a detectable moiety Z in frame with the target protein.

Suitable detection means can include, but are not limited to, use of fluorescent proteins, antibodies against the detectable moiety, pH-sensitive probes, fluorophore binders and enzymatic detection (peroxydase, alkaline phosphatase).

In a preferred embodiment, the target protein Y is fused to a fluorescent protein, such as Green Fluorescent Protein (GFP) and the red fluorescent protein mCherry. Advantageously, this embodiment enables to follow the target protein Y in real-time in living cells.

In another embodiment, the target protein Y is fused to Horse Radish Peroxidase (HRP). Advantageously, this embodiment enables the electron microscopy observation of transport intermediates. It falls within the ability of the skilled person in the art to select the appropriate detection moiety according to the specific goal which is sought.

As used herein, the term “host cell” refers to any eukaryotic cell which can be genetically manipulated to express the first and second fusion proteins of the invention. Typically, the host cell according to the invention can be a yeast cell or an insect cell or a mammalian cell, such as a rodent or primate or human cell. Preferably, the host cells are HeLa and RPE-1 cell lines of human origin. According to the present invention, the host cell can be an in vitro host cell, in culture, or an in vivo host cell, within a living organism.

The method of the invention can be carried out in any cellular model, of any origin and at any physiological temperature imposed by the chosen host owing that: (1) one can find proteins stably localised in the chosen donor compartment that can be used as a Hook and (2) the host cell allows interaction of the interaction domains and is permeant to the Ligand molecule.

General method according to the invention:

In the two-component system of the invention, both the first fusion protein A-X (HOOK) and the second fusion protein B-Y (REPORTER) need to be expressed in the same host cell.

This can be achieved by a variety of modes among which:
transfection of cells using two separate plasmids
single transfection using a plasmid bearing a bicistronic expression cassette.

Alternatively, instead of using a plasmid, viral delivery can also be used. Alternatively, stable cell lines expressing one or both constructs (using single or multiple expression vectors) can also be generated, according to general procedures in the art.

Keeping the cells at their physiological temperature, the reporter is blocked in the hook-containing compartment. When using the interaction-by-default embodiment, this will naturally occur. However, when using the molecular-dependant embodiment, the ligand that acts as a bridging molecule and ensures the interaction of the two domains A and B has to be added at this step. The skilled person in the art will be able to establish the time necessary to block all the molecules of reporter in the donor compartment without excessive experimentation.

Typically, the time necessary to block all the molecules of reporter in the donor compartment can be comprised between 2 and 24 h, preferably between 6 and 16 h, even more preferably about 16 h (overnight).

Typically, approximately 6 hours are sufficient to block Golgi enzymes in the ER. To start measuring the secretion of reporter, the block is released. When using the [ID]-RUSH, the ligand L will be added at this step, when using the [MD]-RUSH the bridging molecule ligand L will be washed out and the competitor C added if necessary.

In one embodiment, the method of the invention can be used to identify conditions or molecules that perturb the trafficking of the target protein. For example, the method of the invention can be used to screen for compounds that perturb the trafficking of the target protein.

Typically, said compounds can be siRNA. For example, the method of the invention can be used to screen a siRNA library, available from many providers (Qiagen, Thermo, Sigma-Proligo), to inactivate a large diversity of regulatory genes.

Typically, said compounds can be small molecules such as molecules of a chemical drug library. These libraries are available from many providers such as ChemBridge, Prestwick Chemical or MayBridge.

The method and kit of the invention can also be used for in vivo applications, such as regulating the transport of a normal or mutated growth factor (e.g. EGF, VEGF), hormones (Insulin, Prolactin) or their receptors or of a pathological molecule (amyloid peptide), with a tight control in time. Examples of the use of such animal models include the production of tumour development at later stage during animal life, development defects of physiological alteration that mimics human diseases.

The invention will be further described by the following figures and examples, which are not intended to limit the scope of the protection defined by the claims.

### FIGURE LEGENDS

FIG. 1: general schemes of the RUSH system.

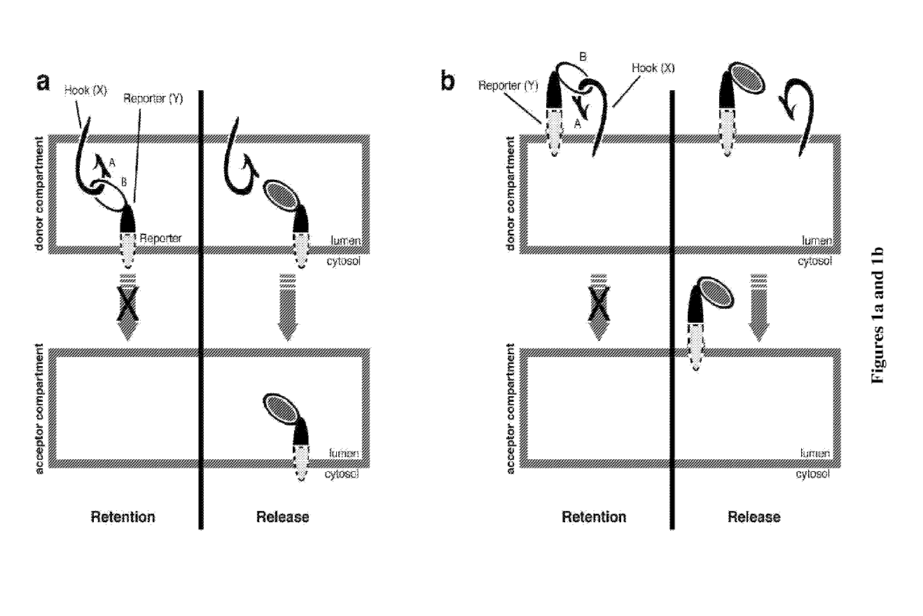

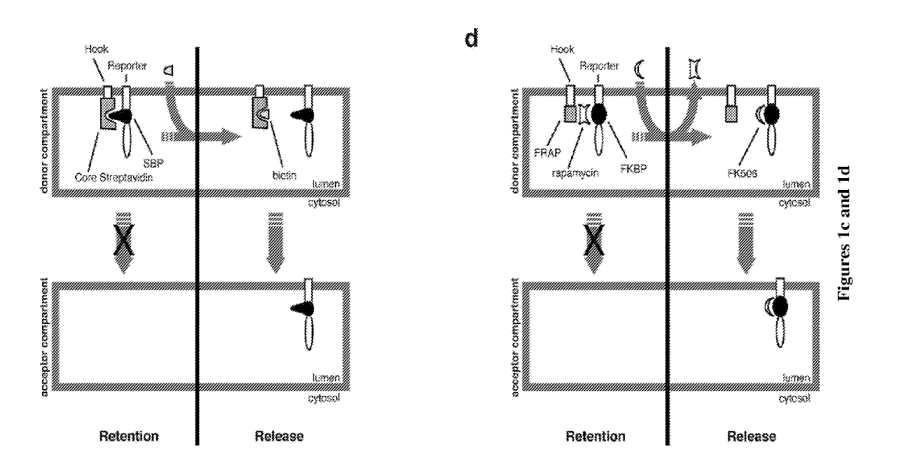

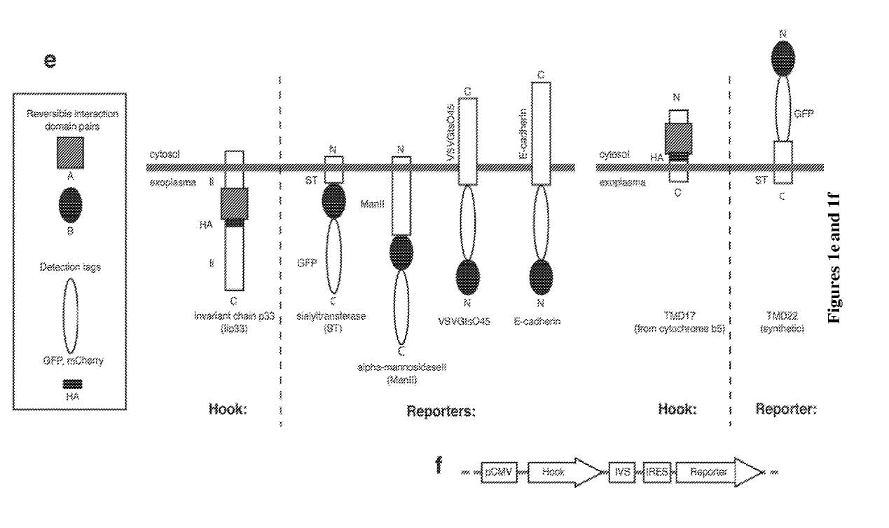

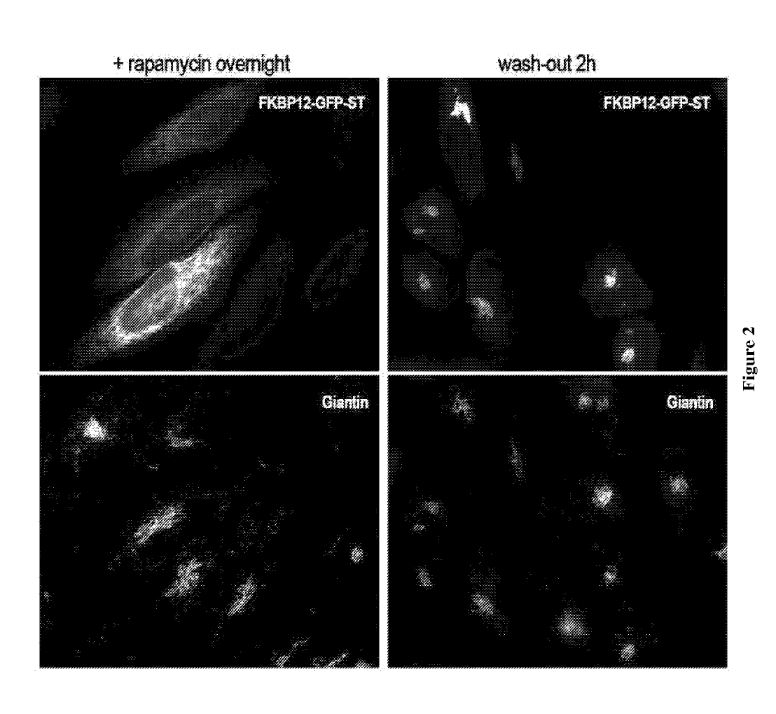

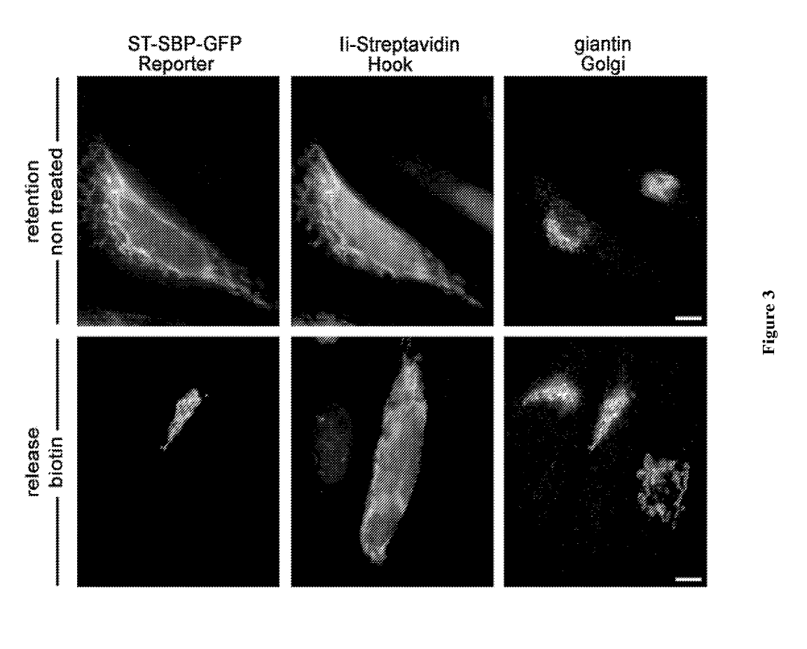

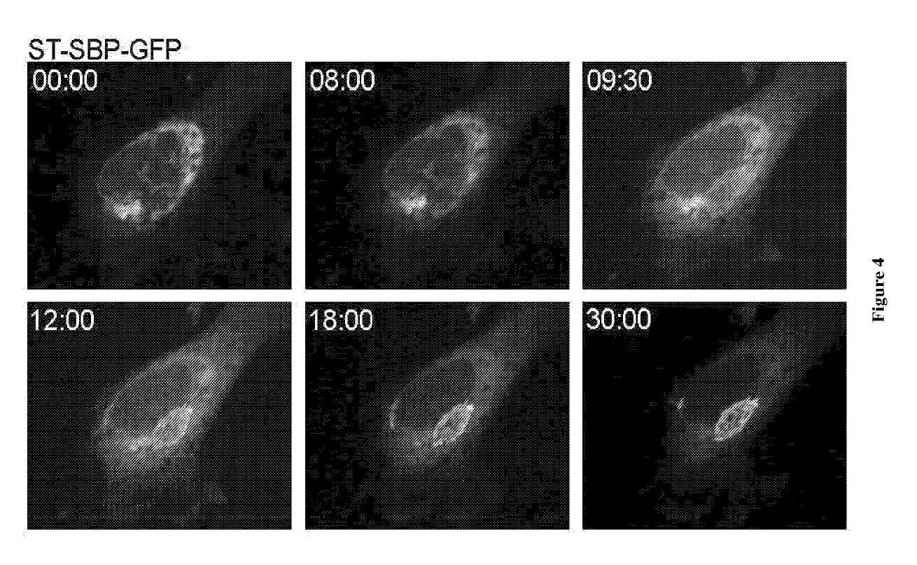

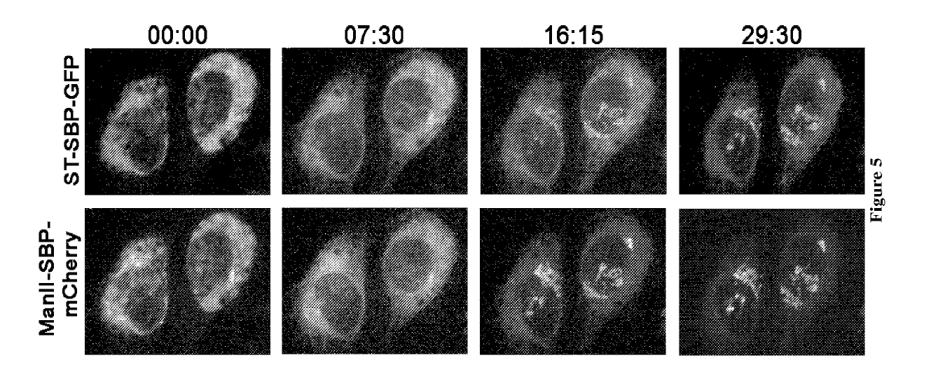

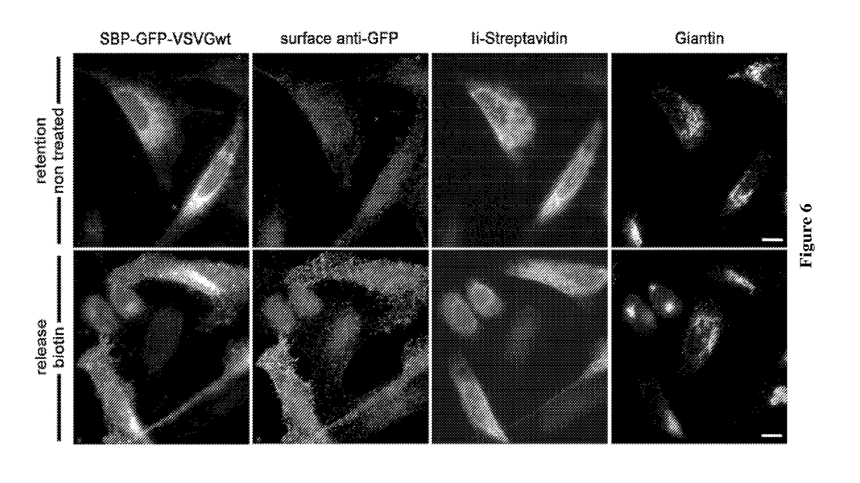

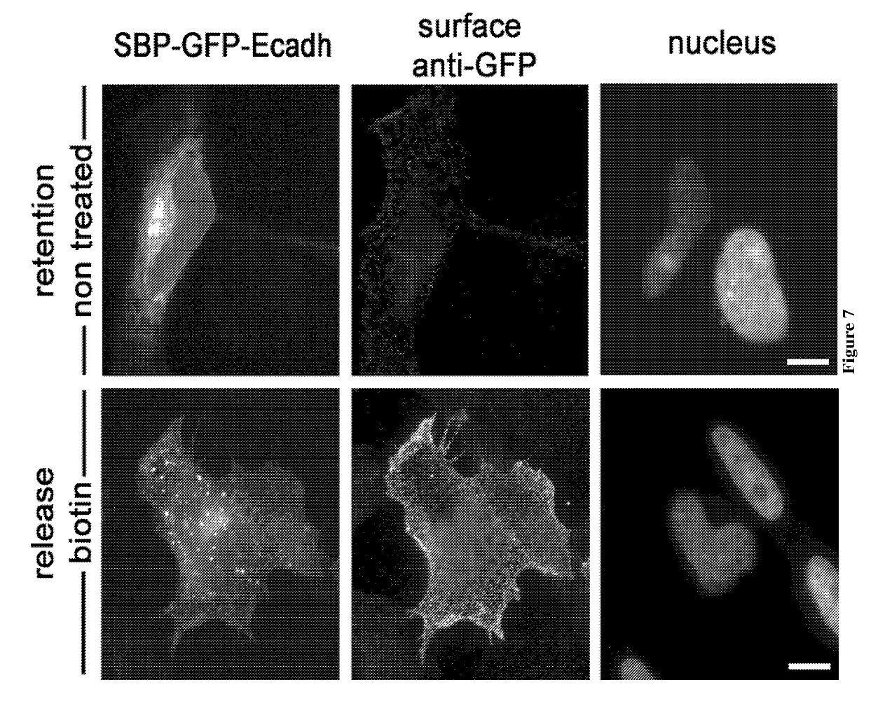

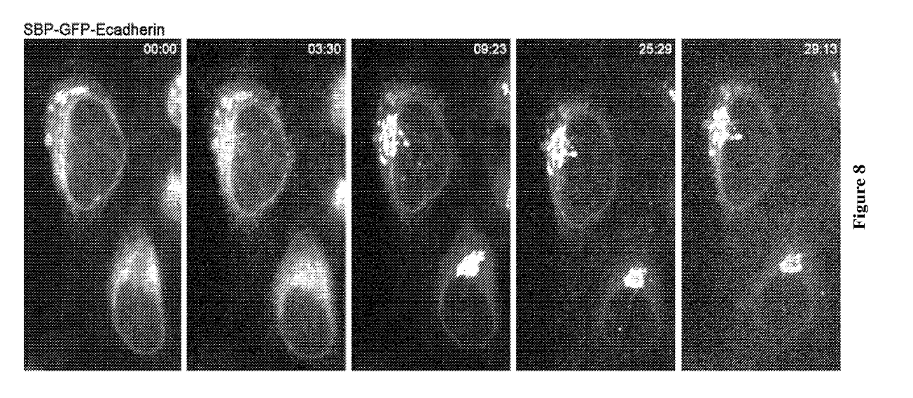

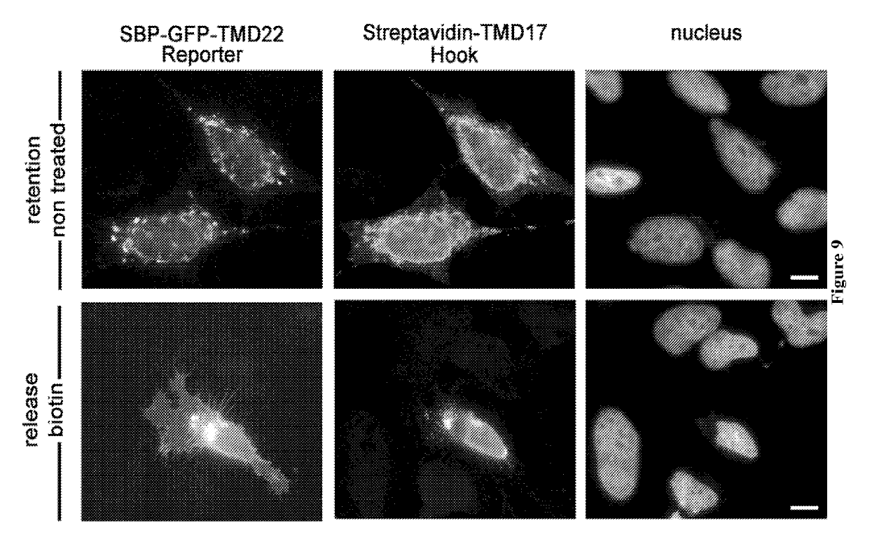

a, b: The two topologies of the RUSH system. The RUSH system is a two-state secretory assay. In one condition (“retention”), the reporter protein B-Y is stably kept in a donor compartment by a Hook protein A-X through the specific interaction of two domains A and B respectively fused to the target protein Y and to the retention domain X. In the second condition (“release”), the interaction between the two domains is reverted and the reporter is released, free to follow its natural trafficking pathway. The interaction domains can be located in the lumen of the compartment (RUSH^{L}, a) or in the cytoplasmic face (RUSH^{C}, b).

c, d: The two reversible interaction set-ups of the RUSH system. The reversible interaction of the hook and the reporter protein can be due to an interaction by default (RUSH ID), or to a molecule-dependent interaction (RUSH MD). An example of the ID mode is shown in c where the reporter displays a streptavidin domain that interacts by default with the SBP tag. Upon addition of biotin, this interaction is competed out and the reporter is free to get transported. In d, the hook is fused to a FRAP domain that interacts with a FKBP12 domain fused to the reporter molecule. This interaction only occurs in the presence of rapamycin. Upon removal of rapamycin (and competition with FK506 to accelerate the release), the interaction is reverted and the reporter is free to get transported.

e: some examples of Hooks and Reporters.

f: schematic representation of the bicistronic constructs used in the Examples.

FIG. 2: Analysis of the trafficking of the Golgi enzyme ST using the reversible interaction between FKPB12 and FRAP (RUSH^{L}[MD]).

The Reporter FKBP12-GFP-ST is retained in the ER in cells expressing the hook Ii-FRAP and in the presence of Rapamycin (left panel). Upon Rapamycin wash-out in the presence of the competitor FK506, the reporter is released and reaches its target Golgi compartment (right panel). The Reporter is visualized using GFP as a detection domain. The target compartment, the Golgi, is stained using anti-Giantin antibodies.

FIG. 3: Analysis of the trafficking of the Golgi enzyme ST using the reversible interaction between core streptavidin and the SBP tag (RUSH^{L}[ID]).

The Reporter ST-SBP-GFP is retained in the ER in cells expressing the hook Ii-Core streptavidin (right panel). Upon biotin addition, the reporter is released and reaches its target Golgi compartment (left panel). The Reporter is visualized using GFP as a detection domain. The target compartment, the Golgi, is stained using anti-Giantin antibodies.

FIG. 4: Time-lapse analysis of the trafficking of the Golgi enzyme ST using the reversible interaction between core streptavidin and the SBP tag (RUSH^{L}[ID]).

The Reporter ST-SBP-GFP is retained in the ER in cells expressing the hook Ii-Core streptavidin. Biotin is added at time 00:00 (min sec) and the release of the reporter is followed by time-lapse fluorescent imaging using a spinning disk equipped confocal microscope at 37° C. The Reporter starts to be visible in the Golgi apparatus in a very short time (9:30) and labels massively the Golgi apparatus by 30 minutes.

FIG. 5: Time-lapse analysis of the trafficking of the Golgi enzymes ST and ManII using the reversible interaction between core streptavidin and the SBP tag (RUSH^{L}[ID]).

The Reporters ST-SBP-GFP (detected using its green fluorescence) and ManII-SBP-mCherry (detected using its red fluorescence) are both retained in the ER in cells expressing the hook Ii-Core streptavidin.

Upon addition of biotin they both reach the Golgi apparatus and can both be followed in real time.

FIG. 6: Analysis of the trafficking of the viral glycoprotein VSV-G using the reversible interaction between core streptavidin and the SBP tag (RUSH^{L}[ID]).

The Reporter SBP-GFP-VSV-G is retained in the ER in cells expressing the hook Ii-Core streptavidin (right panel). The fraction of GFP-tagged VSV-G expressed at the cell surface is labelled using an antibody directed against GFP in the absence of cell permeabilization (surface anti-GFP). The Hook is stained using an anti-Ii monoclonal antibody and the Golgi complex is labelled using an anti-Giantin antibody. Upon biotin addition, the reporter is released and reaches its target plasma membrane compartment (left panel). The Reporter is visualized using GFP as a detection domain. While only traces of the reporter are visible at the cell surface in the retained state, a very large quantity is expressed upon release.

FIG. 7: Analysis of the trafficking of the plasma membrane protein E-Cadherin using the reversible interaction between core streptavidin and the SBP tag (RUSH^{L}[ID]).

The Reporter SBP-GFP-Ecadherin is retained in the ER in cells expressing the hook Ii-Core streptavidin (right panel). The fraction of GFP-tagged E-Cadherin expressed at the cell surface is labelled using an antibody directed against GFP in the absence of cell permeabilization (surface anti-GFP). Upon biotin addition, the reporter is released and reaches its target plasma membrane compartment (left panel). The Reporter is visualized using GFP as a detection domain. While only traces of the reporter are visible at the cell surface in the retained state, a very large quantity is expressed upon release.

FIG. 8: Time-lapse analysis of the trafficking of the plasma membrane protein E-Cadherin using the reversible interaction between core streptavidin and the SBP tag (RUSH^{L}[ID]).

The Reporter SBP-GFP-Ecadherin is retained in the ER in cells expressing the hook Ii-Core streptavidin. Biotin is added at time 00:00 (min:sec) and the release of the reporter is followed by time-lapse fluorescent imaging using a spinning disk equipped confocal microscope at 37° C. The Reporter is visualized using GFP as a detection domain. Significant quantities of the reporter are visible in the Golgi apparatus from 03:30 and continue to increase. The reporter starts to be visible at the plasma membrane around 30:00. Note that transport intermediates (in the form of punctuate staining) are visible at early (ER to Golgi) and late (Golgi to plasma membrane) time points.

FIG. 9: Analysis of the trafficking of the synthetic plasma membrane protein TMD22 using the reversible interaction between core streptavidin and the SBP tag (RUSH^{c}[ID]).

The Reporter SBP-GFP-TMD22 is retained in the ER in cells expressing the hook TMB 17-streptavidin (right panel). In this set-up (RUSH^{C}) the retention domains are located in the cytoplasmic portions of the hook and of the reporter. Upon biotin addition, the reporter is released and reaches its target plasma membrane compartment (left panel). The reporter is visualized using GFP as a detection domain. While only traces of the reporter are visible at the cell surface in the retained state, a very large quantity is expressed upon release.

### EXAMPLES

### Material and Methods

### Reporter Assay Constructions (See Table 1)

### Development of an IRES Vector:

The RUSH system necessitates the simultaneous presence of both a Hook protein and of a Reporter protein in the same cell.

While co-transfection or co-infection could be used we first developed an IRES (Internal Ribosome Entry Site)-based vector to allow simultaneous expression. The Hook is inserted before and the Reporter after the IRES to ensure that enough Hook will be expressed to retain every reporter molecule.

The IRES Vector is based on the pIRESneo3 (Clontech-Takara Bio Europe, Saint-Germain-en-Laye, France). The Hook is inserted using the MCS of the vector. To insert the reporter we modified the vector by replacing the Neo cassette by a Multi-Cloning Site containing the 8-base cutter recognition sites AscI, SfiI and PacI.

Construction of the RUSH^{L} Ii-FRAP [HOOK]/[REPORTER] pIRES vector:

As a first validation we implemented a pair of proteins already used by Dr V. Malhotra {Cell and Developmental Biology Department of University of California San Diego}, (Pecot, 2004; Pecot, 2006) from whom we obtained the source sequences. The Hook is the Invariant chain Iip33 that cannot escape the ER due to a double arginine signal. It is tagged with the HA epitope and fused to the Rapamycin-binding domain (AA 2026-2114) of the FRAP protein.

The first reporter used was a ST-FKBP-GFP construct and was cloned in the AscI and SfiI sites. It consists of the Golgi localization domain of the Sialyl-transferase (ST) fused to the FK506 binding protein (FKBP) followed by the green Fluorescent protein (GFP).

Other Golgi enzyme reporters or secretory markers were similarly sub-cloned and used as Reporters.

Based on the same construct, we fused the VSVG protein sequence to GFP and FKBP12.

Construction of the RUSH^{L} Streptavidin/SBP-based [HOOK]/[REPORTER] system in the pIRES vector:

The FKBP12 and FRAP domains of the Ii-FRAP/ST-FKBP12 couple are replaced by the Core streptavidin and SBP domains. The two configuration (1) Ii-Streptavidin/ST-SBP and (2) Ii-SBP/ST-Streptavidin are constructed and evaluated. Configuration (1) has the advantage of tagging the reporter molecule with a small tag while configuration (2) because it tag the reporter with Streptavidin has the advantage to offer the opportunity to potentially label the reporter with fluorescent biotin during the release. Replacing FRAP by Streptavidin is done using synthetic genes ready to be inserted using the same restriction enzymes. Replacing FKBP12 by SBP Tag is done using a PCR amplified SBP Tag inserted in the EcoRI-SbfI sites. The whole cassette containing the Hook, the IRES and the Reporter is then cloned in the MfeI-AgeI sites of a pEGFP-C1 vector.

Based on the same construct, we fused VSVG and E-Cadherin to GFP and SBP Tag.

TABLE 1
Constructs RUSH^{L}

| | | Example 1 | Example 2 |
|---|---|---|---|
| | | (“MD” mode) | (“ID” mode) |
| Interaction couple | A | FRAP | Streptavidin |
| | B | FKBP | SBP |
| Retention domain | X | Iip33 | |
| or Hook | | | |
| Target protein | Y | ST | |
| | | ManII | |
| | | VSV-G | |
| | | E-Cadherin | |
| Detectable moiety | Z | GFP | |
| | | mCherry | |
| Ligand | L | Rapamycin | Biotin |
| Competitor | C | FK506 | / |

TABLE 2
Constructs RUSH^{C}

| | | Example 3 |
|---|---|---|
| | | (“ID” mode) |
| Interaction couple | A | Streptavidin |
| | B | SBP |
| Retention domain or Hook | X | TMD17 |
| Target protein | Y | TMD22 |
| Detectable moiety | Z | GFP |
| | | mCherry |
| Ligand | L | Biotin |
| Competitor | C | / |

Construction of the RUSH^{C} Ii-FRAP [HOOK]/[REPORTER] pIRES vector:

In this system the FRAP domain is fused to a myc tag and to the hook sequence. This hook consists of a 17 AA long transmenbrane domain of the rat cytochrome b5 (TMD17). Indeed, it has been shown that this domain fused to the GFP protein is able to mediate the retention in the ER of the GFP (Bulbarelli et al., 2002). The FRAP-myc-TMD17 has been prepared as a synthetic gene (Genescript Inc) and was cloned at the 5′ of the IRES in the NheI-BamHI sites of the vector MCS. The reporter part is cloned after the IRES in the AscI-SfiI sites. A second synthetic gene composed of the FKBP domain fused to a 22 AA long transmembrane domain of the rat cytochrome b5 (TMD22) was prepared (Genescript Inc). Sbfi-FseI sites were added between FKBP and TMD22 to allow the insertion of GFP. This TMD22 domain has been shown to be able to target the GFP to the plasma membrane (Bulbarelli et al., 2002).

Construction of the RUSH^{C} Streptavidin/SBP-based [HOOK]/[REPORTER] system in the pIRES vector:

In the hook part, the FRAP domain is replaced by Core streptavidin which was amplified by PCR and inserted in the NheI-AgeI sites. In the reporter part, the FKBP12 is replaced by the SBP Tag sequence that was amplified by PCR and inserted in the EcoRI-SbfI sites.

### Reagents

Rapamycin (Sigma-Aldrich) was diluted as a stock solution (in Ethanol) at 200 mM final. The stock solution was diluted 1000 times and then 64 times, both in medium, to obtain a final molarity of 3.1 nM. At each step of dilution, the solution was strongly vortexed.

FK506 (Cayman) was diluted in DMSO to obtain a stock solution at 24.8 mM. The stock solution was diluted 50 times in DMSO at room temperature (RT), extensively vortexed, and then diluted again 120 times in medium at RT, vortexing strongly, to obtain a final molarity of 4.1 μM. This final dilution was warmed at 37° C. for a few minutes, then vortexed again before being added to the cells.

D-Biotin (Sigma) is prepared as a stock solution in water at 0.2 mg/mL (0.8 mM). Concentration ranging between 80 μM and 100 nM, and preferably 10 μM, are used to release the reporter from the hook. Culture medium containing no or very low levels of Biotin (equal or less than 0.2 μM) are used.

### Cell Culture and Transfection:

Hela cells were grown at 37° C. in DMEM (Invitrogen) supplemented with L-glutamine, Sodium Pyruvate and 10% Fetal Calf Serum. For transient transfection, cells were plated on coverslips in 150-mm culture dishes and transfected with 25 μg of the plasmid hook-IRES-reporter using the calcium phosphate precipitation method, in presence of 25 mM HEPES. After 4 hours, cells were washed out with fresh medium and incubated overnight in presence of 3.1 nM Rapamycin (Sigma). Then cells were washed out 3 times with medium, and incubated in prewarmed medium with 3.3 μM FK506 (Cayman) and 100 mg/mL cycloheximide (CHX) for several time points. For each time point, 1 mL of medium containing FK506 and CHX was added into a well of a 12-well plate. Then cells were fixed with 4% PAF and processed for fluorescent microscopy. In the streptavidin/SBP set-up, transfection is done similarly but no addition is done when washing the cells after transfection. After 1-hours, biotin (10 μM) is added to induce reporter release. Then cells are then processed as in the MD set-up.

### Immunofluorescence:

Cells were fixed in paraformaldehyde 3% for 15 min, washed in PBS (Phosphate Buffer Saline) and free aldehydes were quenched in NH₄Cl 50 mM for 5 min. Cells were then permeabilized in PBS containing Bovine Serum Albumine (BSA 0.5%) and Saponine (Sapo 0.05%) [PBS/BSA/Sapo] for 20 min and incubated with primary antibodies (human anti-Giantin, Nizak et al. 2003, Moutel et al., 2009) in the same buffer for 30 min Cells were washed in PBS and incubated in PBS/BSA/Sapo with fluorescently labelled secondary antibodies (Jackson Immunoresearch) for 20 min Nuclei were counter-stained with DAPI (4′,6-Diamidino-2-phenylindole, Sigma-Aldrich). Cells were finally washed in PBS before being mounted in Mowiol (Sigma-Aldrich) and observed by fluorescent microscopy.

### Example 1

### Molecule-Dependant Interaction Between FRAP and FKBP12 and Trafficking of a Golgi Enzyme, Sialyl-Transferase (FIG. 2)

### Summary

### Hook Construct: Ii-FRAP-HA

### Reporter Construct: Sialyl Transferase-FKBP12-GFP

In this example, the Hook is based on a variant of the Invariant Chain that cannot move out from the ER. It is fused to the rapamycin-binding protein FRAP to form a first fusion protein and to a HA tag for immunostaining. The reporter is the targeting sequence of a Golgi enzyme sequence (sialyl transferase) fused to the rapamycin- and FK506-binding protein FKBP12 to form the second fusion protein. To follow its trafficking, the reporter has also been fused to a fluorescent GFP protein. The donor compartment is the ER and the target compartment is the Golgi apparatus. Both reporter and hook are expressed under the control of a single promoter.

Retention of the reporter in the ER occurred in the presence of rapamycin. Upon wash-out of rapamycin, and in the presence of FK506 to compete with rapamycin binding to FKBP12, the reporter was released and trafficked toward its target Golgi compartment.

### Protocol and Results

Cells were incubated overnight with low rapamycin concentrations to induce a stable interaction between the FRAP and FKBP12 domains. In these conditions, as shown by immuno-fluorescence, FKBP12-GFP-ST could not reach the Golgi apparatus labeled using a Giantin antibody (FIG. 2, top panel, retained state).

Upon rapamycin wash-out in the presence of FK-506 (FIG. 2, lower panel, released state), the reporter molecules could efficiently exit the ER and reach the Golgi apparatus.

Thus, the RUSH system provides a method for synchronizing the intracellular trafficking of a target protein Y (in the present case ST) in a host cell using two fusion proteins. The first fusion protein X-A serves as a Hook and is able to retain the second fusion protein B-Y in the endoplasmic reticulum in the presence of rapamycin as a ligand L. Upon rapamycin wash-out and addition of a competitor C, the reporter B-Y can be seen to exit the ER and to transit towards the Golgi.

This system is based on the reversible interaction of FRAP and FKBP12 in the presence or absence of rapamycin.

### Example 2

### Interaction by Default Between Streptavidin/SBP Tag and Trafficking of a Golgi Enzyme, Sialyl-Transferase (FIGS. 3 and 4)

### Summary

### Hook: Ii-FRAP

### Reporter: Sialyl Transferase-FKBP12-GFP

In this example, the Hook is based on a variant of the Invariant Chain that cannot move out from the ER. It is fused to the core streptavidin to form a first fusion protein and to a HA tag for immunostaining. The reporter is the targeting sequence of a Golgi enzyme sequence (sialyl transferase) fused to the streptavidin-interacting SBP peptide to form the second fusion protein. To follow its trafficking, the reporter has also been fused to a fluorescent GFP protein. The donor compartment is the ER and the target compartment is the Golgi apparatus. Both reporter and hook are expressed under the control of a single promoter.

Retention of the reporter in the ER occurred by default due to the interaction between SBP and core streptavidin. Upon addition of biotin the reporter was released and trafficked toward its target Golgi compartment.

### Protocol and Results

A hook was constructed using the ER localized Ii33 fused to monomeric Streptavidin (SEQ ID NO:3) (FRAP of the construct from Example 1 is replaced by monomeric Streptavidin). The reporter was the Golgi localization domain of Sialyl Transferase as used in Example 1. The Hook and reporter were co-expressed in cells and the two domains, streptavidin and SBP-tag interact by default, preventing ST transport to the Golgi apparatus. Release of ST was achieved using moderate concentration of free biotin (around 1-10 μM).

In this example, the ID mode of the RUSH system is illustrated.

In the absence of ligand, the interaction between SBP-Tag and Streptavidin occurred and the reporter was trapped in the ER. Upon addition of the ligand, biotin, the interaction was disrupted. The reporter protein (here the targeting domain of ST) was released and could leave the ER in order to traffic towards its target compartment; the Golgi (FIG. 3).

The kinetics observed with this system were extremely fast, suggesting that the inhibition of the interaction between Streptavidin and SBP was not a limiting step. Indeed, as shown in FIG. 4, staining was observed in the Golgi after only a few minutes, and most if not all of the Reporter molecules had reached the Golgi compartment by 18 to 30 minutes after the release.

This provides a unique way to study quantitatively and kinetically, describe molecularly and potentially perturb the traffic of a Golgi enzyme.

### Example 3

### Interaction by Default Between Streptavidin/SBP Tag and Trafficking of a Golgi Enzyme Mannosidase II (FIG. 5)

As in Example 2 but using Mannosidase II targeting domain as a reporter molecule. This example provides another sort of Golgi enzyme to be analyzed. By fusing it to a red fluorescent protein, it was possible to observe two Golgi enzymes (or a Golgi enzyme and another cargo) at the same time and between the same donor and acceptor compartments.

### Example 4

### Interaction by Default Between Streptavidin/SBP Tag and Trafficking of a Viral Protein VSV-G (FIG. 6)

As in example 2 but using the viral glycoprotein VSV-G as a reporter molecule. This is a very classical reporter usually used in its thermosensitive version to study and quantify traffic between the ER and the plasma membrane. Using the RUSH system, the same analysis was performed but without the use of temperature block. Cells can thus be studied at their normal, physiological, temperature.

### Example 5

### Interaction by Default Between Streptavidin/SBP Tag and Trafficking of a Plasma Membrane Protein E-Cadherin (FIGS. 7 and 8)

As in example 2 but using the adhesion molecule E-Cadherin as reporter molecule. This example shows that transport from the ER to the plasma membrane could be followed synchronously and in real time. Time lapse analysis allowed identification of transport intermediates (ER-to-Golgi and Golgi-to-Plasma membrane) (FIG. 8). The RUSH system can thus be used to study quantitatively and kinetically, describe molecularly and potentially perturb the traffic of a plasma membrane localized protein.

### Example 6

### Interaction by Default Between Streptavidin/SBP Tag in Cytoplasmic Topology (FIG. 9).

### Summary

Hook: TMD17 (cytochrome b5)

### Reporter: TMD22-GFP

In this example, the Hook is based on the transmembrane domain of the cytochrome b5 that behaves as a resident protein of the ER. It is fused to the core streptavidin to form a first fusion protein and to a mys tag for immunostaining. The reporter is a synthetic transmembrane domain, based on cytochrome b5 and that traffics toward the plasma membrane fused to the streptavidin-interacting SBP peptide to form the second fusion protein. To follow its trafficking, the reporter has also been fused to a fluorescent GFP protein. The donor compartment is the ER and the target compartment is the plasma membrane. Both reporter and hook are expressed under the control of a single promoter.

Retention of the reporter in the ER occurs by default due to the interaction between SBP and core streptavidin. Upon addition of biotin the reporter is released and traffics toward its target Golgi compartment. This set-up (RUSH^{c}) allows retention and release of cargo from the cytoplasmic face of the membrane.

### Protocol and Results

As an example of the RUSH^{C} set-up we used the transmembrane domain of the cytochrome B5 (TMD17) as a hook and a longer synthetic domain based on cytochrome b5, TMD22. At steady state the retention was extensive. Upon biotin addition, release was observed and the reporter TMD22 continued its traffic toward the plasma membrane. This shows that protein that do not have any luminal domain, or that cannot be tagged in their luminal part, can also be studied using the RUSH system.

Similar experiments were performed using Giantin as a Hook to retain the protein of interest in the Golgi compartment, rather than the ER.

### REFERENCES

Throughout this application, various references describe the state of the art to which this invention pertains. The disclosures of these references are hereby incorporated by reference into the present disclosure.
Achour L, Scott M G, Shirvani H, Thuret A, Bismuth G, Labbé-Jullié C, Marullo S. 2009. CD4-CCR5 interaction in intracellular compartments contributes to receptor expression at the cell surface. Blood. 113(9):1938-47
Altan-Bonnet, N., R. Sougrat, and J. Lippincott-Schwartz. 2004. Molecular basis for Golgi maintenance and biogenesis. Curr Opin Cell Biol. 16:364-72.
Amaral M D 2005. Processing of CFTR: traversing the cellular maze—how much CFTR needs to go through to avoid cystic fibrosis? Pediatr Pulmonol. 2005 June; 39(6):479-91
Arnheiter, H., M. Dubois-Dalcq, and R. A. Lazzarini. 1984. Direct visualization of protein transport and processing in the living cell by microinjection of specific antibodies. Cell. 39:99-109.
Bard, F., L. Casano, A. Mallabiabarrena, E. Wallace, K. Saito, H. Kitayama, G. Guizzunti, Y. Hu, F. Wendler, R. Dasgupta, N. Perrimon, and V. Malhotra. 2006. Functional genomics reveals genes involved in protein secretion and Golgi organization. Nature. 439:604-7.
Barr, F. A. 2004. Golgi inheritance: shaken but not stirred. J Cell Biol. 164:955-8. Clackson, T. 1997. Controlling mammalian gene expression with small molecules. Curr. Opi. Chem. Biol. 1:210-218.
Brown, E. J., M. W. Albers, T. B. Shin, K. Ichikawa, C. T. Keith, W. S. Lane, and S. L. Schreiber. 1994. A mammalian protein targeted by G1-arresting rapamycin-receptor complex. Nature. 369:756-8.
Bulbarelli, A., T. Sprocati, M. Barberi, E. Pedrazzini, and N. Borgese. 2002. Trafficking of tail-anchored proteins: transport from the endoplasmic reticulum to the plasma membrane and sorting between surface domains in polarised epithelial cells. Journal of cell science. 115:1689-702.
Cosson, P., M. Amherdt, J. E. Rothman, and L. Orci. 2002. A resident Golgi protein is excluded from peri-Golgi vesicles in NRK cells. PNAS:12831-4.
Fu, J., G. Pirozzi, A. Sanjay, R. Levy, Y. Chen, C. De Lemos-Chiarandini, D. Sabatini, and G. Kreibich. 2000. Localization of ribophorin II to the endoplasmic reticulum involves both its transmembrane and cytoplasmic domains. Eur J Cell Biol. 79:219-28.
Glick, B. S. 2002. Can the Golgi form de novo? Nat Rev Mol Cell Biol. 3:615-9. Harris D A. Trafficking, 2003. Turnover and membrane topology of PrP. Br Med Bull. 66:71-85.
Hennecke, S., and P. Cosson. 1993. Role of transmembrane domains in assembly and intracellular transport of the CD8 molecule. J Biol Chem. 268:26607-12.
Keller, P., D. Toomre, E. Diaz, J. White, and K. Simons. 2001. Multicolour imaging of post-Golgi sorting and trafficking in live cells. Nat Cell Biol. 3:140-9.
Kreis, T. E. 1986. Microinjected antibodies against the cytoplasmic domain of vesicular stomatitis virus glycoprotein block its transport to the cell surface. The EMBO journal. 5:931-41.
Ladinsky, M. S., D. N. Mastronarde, J. R. McIntosh, K. E. Howell, and L. A. Staehelin. 1999. Golgi structure in three dimensions: functional insights from the normal rat kidney cell. J Cell Biol. 144:1135-49.
Lafay, F. 1974. Envelope proteins of vesicular stomatitis virus: effect of temperature-sensitive mutations in complementation groups III and V. J. Virol. 14:1220-8.
Maecker, H. T., S. C. Todd, and S. Levy. 1997. The tetraspanin superfamily: molecular facilitators. FASEB J. 11:428-42.
Maki, N., F. Sekiguchi, J. Nishimaki, K. Miwa, T. Hayano, N. Takahashi, and M. Suzuki. 1990. Complementary DNA encoding the human T-cell FK506-binding protein, a peptidylprolyl cis-trans isomerase distinct from cyclophilin Proc Natl Acad Sci USA. 87:5440-3.
Martinez-Menarguez, J. A., R. Prekeris, V. M. Oorschot, R. Scheller, J. W. Slot, H. J. Geuze, and J. Klumperman. 2001. Peri-Golgi vesicles contain retrograde but not anterograde proteins consistent with the cisternal progression model of intra-Golgi transport. J Cell Biol. 155:1213-24.
Mironov, A. A., G. V. Beznoussenko, R. S. Polishchuk, and A. Trucco. 2005. Intra-Golgi transport: A way to a new paradigm? Biochim Biophys Acta. 1744:340-50.
Moutel, S., A. El Marjou, O. Vielemeyer, C. Nizak, P. Benaroch, S. Dubel, and F. Perez. 2009. A multi-Fc-species system for recombinant antibody production. BMC Biotechnol. 9:14.
Nizak, C., S. M. Martin-Lluesma, S. Moutel, A. Roux, T. E. Kreis, B. Goud, and F. Perez. 2003. Recombinant antibodies selected against subcellular fractions to track endogenous protein dynamics in vivo. Traffic. 7:739-753.
Patterson, G. H., K. Hirschberg, R. S. Polishchuk, D. Gerlich, R. D. Phair, and J. Lippincott-Schwartz. 2008. Transport through the Golgi apparatus by rapid partitioning within a two-phase membrane system. Cell. 133:1055-67.
Pecot, M. Y., and V. Malhotra. 2004. Golgi membranes remain segregated from the endoplasmic reticulum during mitosis in mammalian cells. Cell. 116:99-107.
Pecot, M. Y., and V. Malhotra. 2006. The Golgi apparatus maintains its organization independent of the endoplasmic reticulum. Molecular biology of the cell. 17:5372-80.
Pelham, H. R. 2001. Traffic through the Golgi apparatus. J Cell Biol. 155:1099-101.
Presley, J. F., N. B. Cole, T. A. Schroer, K. Hirschberg, K. J. Zaal, and J. Lippincott-Schwartz. 1997. ER-to-Golgi transport visualized in living cells. Nature. 389:81-5.
Prydz, K., G. Dick, and H. Tveit. 2008. How Many Ways Through the Golgi Maze?
Puri, S., and A. D. Linstedt. 2003. Capacity of the Golgi Apparatus for Biogenesis from the Endoplasmic Reticulum. Mol Biol Cell. 14:5011-5018.
Puthenveedu, M. A., and A. D. Linstedt. 2005. Subcompartmentalizing the Golgi apparatus. Curr Opin Cell Biol. 17:369-375.
Rabouille, C., and J. Klumperman. 2005. Opinion: The maturing role of COPI vesicles in intra-Golgi transport. Nat Rev Mol Cell Biol. 6:812-7.
Rambourg, A., and Y. Clermont. 1997. Three dimensional structure of the Golgi apparatus in mammalian cells. In The Golgi apparatus. E.G.a.R. Berger, J, editor. Birkhä user, Basel, Switzerland. 37-61.
Rayner J C, Pelham H R. 1997. Transmembrane domain-dependent sorting of proteins to the ER and plasma membrane in yeast. EMBO J. 16(8):1832-41.
Rivera, V. M., X. Wang, S. Wardwell, N. L. Courage, A. Volchuk, T. Keenan, D. A. Holt, M. Gilman, L. Orci, F. Cerasoli, Jr., J. E. Rothman, and T. Clackson. 2000. Regulation of protein secretion through controlled aggregation in the endoplasmic reticulum. Science (New York, N.Y. 287:826-30.
Scales, S. J., R. Pepperkok, and T. E. Kreis. 1997. Visualization of ER-to-Golgi transport in living cells reveals a sequential mode of action for COPII and COPI. Cell. 90:1137-1148.
Schuck S, Simons K. 2006. Controversy fuels trafficking of GPI-anchored proteins. J Cell Biol.; 172(7):963-5.
Schutze, M. P., P. A. Peterson, and M. R. Jackson. 1994. An N-terminal double-arginine motif maintains type II membrane proteins in the endoplasmic reticulum. EMBO J. 13:1696-705.
Shorter, J., and G. Warren. 2002. Golgi architecture and inheritance. Annu Rev Cell Dev Biol. 18:379-420.
Standaert, R. F., A. Galat, G. L. Verdine, and S. L. Schreiber. 1990. Molecular cloning and overexpression of the human FK506-binding protein FKBP. Nature. 346:671-4.
Storrie, B. 2005. Maintenance of Golgi apparatus structure in the face of continuous protein recycling to the endoplasmic reticulum: making ends meet. Int Rev Cytol. 244:69-94.
Strubin, M., E. O. Long, and B. Mach. 1986. Two forms of the Ia antigen-associated invariant chain result from alternative initiations at two in-phase AUGs. Cell. 47:619-25.
Strubin, M., B. Mach, and E. O. Long. 1984. The complete sequence of the mRNA for the HLA-DR-associated invariant chain reveals a polypeptide with an unusual transmembrane polarity. EMBO J. 3:869-72.
Tanya A. Rege, James S. Hagood. 2006. Thy-1, a versatile modulator of signaling affecting cellular adhesion, proliferation, survival, and cytokine/growth factor responses. Biochimica et Biophysica Acta 1763:991-999.
Wilson, D. S., A. D. Keefe, and J. W. Szostak. 2001. The use of mRNA display to select high-affinity protein-binding peptides. Proc. Natl. Acad. Sci. USA. 98:3750-3755.
Wu, S. C., and S. L. Wong. 2005. Engineering soluble monomeric streptavidin with reversible biotin binding capability. J Biol Chem. 280:23225-31.

1-18. (canceled)

19. Method for regulating the intracellular trafficking of a target protein Y in a host cell comprising the steps consisting of:
a) providing a host cell;
b) providing a first expression vector comprising a nucleotide sequence encoding a first fusion protein of formula A-X, wherein A is an interaction domain and X is a domain capable of retaining the first fusion protein of formula A-X in a given intracellular compartment;
c) providing a second expression vector comprising a nucleotide sequence encoding a second fusion protein of formula B-Y, wherein B is an interaction domain and Y is the target protein;
wherein A and B are capable of a conditional interaction according to the presence or absence of a ligand L.

20. A method according to claim 19, wherein the conditional interaction between A and B is disrupted by addition of a ligand L.

21. A kit for regulating the intracellular trafficking of a target protein Y in a host cell comprising:
a first expression vector comprising a nucleotide sequence encoding a first fusion protein of formula A-X wherein A is an interaction domain and X is a domain capable of retaining the first fusion protein of formula A-X in a given compartment and
a second expression vector comprising a nucleotide sequence encoding a second fusion protein of formula B-Y, wherein B is an interaction domain and Y is the target protein;
wherein A and B are capable of a conditional interaction according to the presence or absence of a ligand L.

22. A kit according to claim 21, further comprising a host cell capable of being transformed with said first and second expression vectors.

23. A kit according to claim 21, further comprising a transfection reagent.

24. A kit according to claim 21, further comprising a ligand L.

25. A method according to claim 19, wherein the interaction between A and B occurs only in the presence of the ligand.

26. A method according to claim 19, wherein the interaction between A and B occurs only in the absence of the ligand.

27. A method according to claim 19, wherein B-Y further comprises a detectable moiety Z.

28. A method according to claim 19, wherein said host cell is a eukaryotic host cell selected from the group consisting of a yeast cell, an insect cell and a mammalian cell.

29. A method according to claim 19, wherein A and B are FKPB12 and FRAP or FRAP and FKBP12, respectively.

30. A method according to claim 19, wherein A and B are SBP and Streptavidin or Streptavidin and SBP, respectively.

31. A method according to claim 19, wherein X is selected from the group consisting of an isoform of the invariant chain which resides in the ER (Ii33); Ribophorin I or II; SEC61, cytochrome b5; Giantin; Golgi enzymes such as ManII (α-1,3-1,6 mannosidase), TGN38/46; Menkes receptor; Sialyl Transferase (β-galactosamide α-2,6-sialyltranferase 1), and GalT (β-1,4-galactosyltransferase 1); and GPI-anchored proteins such as Thy-1 and PRNP.

32. A method according to claim 19, wherein Y is selected from the group consisting of growth factors, receptors, plasma membrane markers and Major HistoCompatibility (MHC) molecules, adhesion molecules lysosomal enzymes, Golgi enzymes, viral glycoproteins, tetraspanning proteins, signal transduction proteins; synthetic transmembrane domains; transporter proteins like the multidrug resistance protein ABCB1; GPI-anchored proteins; hormones; hormone receptors and pathological molecules such as amyloid peptide.

33. A method according to claim 19, wherein said first expression vector comprises a nucleotide sequence encoding A and a multiple cloning site enabling the in-frame insertion of a nucleotide sequence encoding X in order to encode a first fusion protein A-X.

34. A method according to claim 19, wherein said second expression vector comprising a nucleotide sequence encoding B and a multiple cloning site for the in-frame insertion of Y in order to encode a second fusion protein of formula B-Y.

35. A method according to claim 19, wherein the first and second vector are a single vector and said single vector comprises a bicistronic expression cassette.

36. A kit according to claim 21, wherein the interaction between A and B occurs only in the absence of the ligand.

37. A kit according to claim 21, wherein A and B are SBP and Streptavidin or Streptavidin and SBP, respectively.

38. A kit according to claim 21, wherein X is selected from the group consisting of an isoform of the invariant chain which resides in the ER (Ii33); Ribophorin I or II; SEC61, cytochrome b5; Giantin; Golgi enzymes such as ManII (α-1,3-1,6 mannosidase), TGN38/46; Menkes receptor; Sialyl Transferase (β-galactosamide α-2,6-sialyltranferase 1), and GalT (β-1,4-galactosyltransferase 1); and GPI-anchored proteins such as Thy-1 and PRNP.


SEQUENCE LISTING (3 sequences)

SEQ ID NO: 1 - protein, 38 aa, Artificial Sequence
  SBP tag
MDEKTTGWRGGHVVEGLAGELEQLRARLEHHPQGQREP

SEQ ID NO: 2 - protein, 160 aa, Artificial Sequence
  Core Streptavidin
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 3 - protein, 160 aa, Artificial Sequence
  Monomeric Core Streptavidin
MDPSKDSKAQVSAAEAGITGTWYNQLGSTFIVTAGADGALTGTYESAVGNAESRYTLTGRYDSAPATDGSGTALGWRVAWKNNYRNAHSATTWSGQYVGGAEARINTQWTLTSGTTEANAWKSTLRGHDTFTKVKPSAASIDAAKKAGVNNGNPLDAVQQ